(19) United States
(12) Patent Application Publication
(10) Pub. No.: US 2026/0248546 A1
(43) Pub. Date: Aug. 27, 2026

(54) ARTICULATING MEMBER WITH CENTER BEAM

(71) Applicant: Boston Scientific Scimed, Inc., Maple Grove, MN (US)

(72) Inventors: David Cory Kirt, Minnetonka, MN (US); Emily Luecke, Osseo, MN (US)

(21) Appl. No.: 19/549,759

(22) Filed: Feb. 25, 2026

Related U.S. Application Data
(60) Provisional application No. 63/764,297, filed on Feb. 27, 2025.

(51) Int. Cl. A61B 18/00

(52) U.S. Cl. CPC A61B 18/00; A61B 2018/00184

(57) ABSTRACT

A medical device includes a handle and a tubular shaft having a proximal portion extending from the handle, and a distal portion having a distal end and a deflection region, the shaft defining a longitudinal axis. The tubular shaft includes an outer tubular jacket and an articulation member disposed within the jacket in the deflection region, the articulation member having a wall and a beam extending across the wall from a first perimetric position to a second perimetric position. The tube defines a plurality of openings, the openings are arranged in a first array and a second array, the openings in the first array are diametrically opposed to the openings in the second array, and the openings in the first array do not circumferentially overlap the beam.

### CROSS REFERENCE TO RELATED APPLICATIONS

This application claims priority to U.S. Provisional Patent Application No. 63/764,297 entitled “ARTICULATING MEMBER WITH CENTER BEAM,” filed February 27, 2025, which is hereby incorporated by reference in its entirety.

### TECHNICAL FIELD

The present invention relates to medical devices and methods for catheters for medical procedures. More specifically, the invention relates to devices and methods that include directional enhancement for catheters such as steerable catheters.

### BACKGROUND

Various medical procedures involve catheters inserted into a patient's vasculature. In certain procedures, the catheter may be navigated through the vasculature to a target location in the body. The distal end of the catheters may be inserted into the patient's heart chambers in, for example, interventional electrophysiology procedures. The distal end of the catheter may include one or more electrodes that are used to delivery therapy (e.g., ablation) or map the surface of the heart tissue (e.g., identify the locations of heart tissue that are a source of the arrhythmias). Steering such a catheter can involve controlled bending at the distal end, which can cause failures of the catheter over time and/or due to challenging anatomy.

### SUMMARY

In Example 1, a medical device comprising: a handle; and a tubular shaft having a proximal portion extending from the handle, and a distal portion having a distal end and a deflection region, the shaft defining a longitudinal axis and including: an outer tubular jacket; and an articulation member disposed within the jacket in the deflection region, the articulation member having a wall and a beam extending across the wall from a first perimetric position to a second perimetric position, wherein: the tube defines a plurality of openings; the openings are arranged in a first array and a second array; the openings in the first array are diametrically opposed to the openings in the second array; and the openings in the first array do not circumferentially overlap the beam.

In Example 2, the medical device of Example 1, wherein the beam bisects a lumen in the articulation member into two sections.

In Example 3, the medical device of Example 2, wherein the two sectors are equally sized.

In Example 4, the medical device of any of Examples 1-3, wherein the beam includes fillets where the beam connects to the wall.

In Example 5, the medical device of Example 4, wherein the openings do not extend into any of the fillets.

In Example 6, the medical device of Example 4, wherein a radius of curvature of the fillets is between one-quarter and four times a thickness of the beam.

In Example 7, the medical device of any of Examples 1-6, wherein the first perimetric position is diametrically opposed to the second perimetric position.

In Example 8, the medical device of any of Examples 1-7, wherein a first surface of the beam is parallel to a second surface of the beam.

In Example 9, the medical device of any of Examples 1-8, wherein the beam comprises two spars with a gap therebetween.

In Example 10, the medical device of any of Examples 1-8, wherein the beam includes a bend such that the beam is connected to the wall at an angle.

In Example 11, the medical device of any of Examples 1-10, wherein the first array is longitudinally offset from the second array.

In Example 12, the medical device of Example 11, wherein the first array is offset such that an opening in the first array is longitudinally positioned halfway between two openings in the second array.

In Example 13, The medical device of any of Examples 1-12, wherein the articulation member includes a first steering wire lumen on a first side of the beam and a second steering wire lumen on a second side of the beam.

In Example 14, the medical device of Example 13, wherein: a. the first steering wire lumen and the second steering wire lumen are positioned in a first plane; b. the beam extends in a second plan; and c. the first plane is orthogonal to the second plane.

In Example 15, the medical device of any of Examples 1-14, wherein an innermost extent of the plurality of openings is parallel to the beam.

In Example 16, a medical device comprising: a. a handle; and b. a tubular shaft having a proximal portion extending from the handle, and a distal portion having a distal end and a deflection region, the shaft defining a longitudinal axis and including a. an outer tubular jacket; and b. an articulation member disposed within the jacket in the deflection region, the articulation member having a wall and a beam extending across the wall from a first perimetric position to a second perimetric position, wherein: c. the tube defines a plurality of openings; d. the openings are arranged in a first array and a second array; e. the openings in the first array are diametrically opposed to the openings in the second array; and f. the openings in the first array do not circumferentially overlap the beam.

In Example 17, the medical device of Example 16, wherein the beam bisects a lumen in the articulation member into two sectors.

In Example 18, the medical device of Example 17, wherein the two sectors are equally sized.

In Example 19, The medical device of Example 16, wherein the beam includes fillets where the beam connects to the wall.

In Example 20, the medical device of Example 19, wherein the openings do not extend into any of the fillets.

In Example 21, the medical device of Example 19, wherein a radius of curvature of the fillets is between one-quarter and four times a thickness of the beam.

In Example 22, the medical device of Example 16, wherein the first perimetric position is diametrically opposed to the second perimetric position.

In Example 23, The medical device of Example 16, wherein a first surface of the beam is parallel to a second surface of the beam.

In Example 24, The medical device of Example 16, wherein the beam comprises two spars with a gap therebetween.

In Example 25, the medical device of Example 16, wherein the beam includes a bend such that the beam is connected to the wall at an angle.

In Example 26, the medical device of Example 16, wherein the first array is longitudinally offset from the second array.

In Example 27, the medical device of Example 26, wherein the first array is offset such that an opening in the first array is longitudinally positioned halfway between two openings in the second array.

In Example28, the medical device of Example 16, wherein the articulation member includes a first steering wire lumen on a first side of the beam and a second steering wire lumen on a second side of the beam.

In Example 29, the medical device of Example 28, wherein: a. the first steering wire lumen and the second steering wire lumen are positioned in a first plane; b. the beam extends in a second plane; and c. the first plane is orthogonal to the second plane.

In Example 30, the medical device of Example 1, wherein an innermost extent of the plurality of openings is parallel to the beam.

In Example 31, a medical device comprising: a. a handle; and b. a tubular shaft having a proximal portion extending from the handle, and a distal portion having a distal end and a deflection region, the shaft defining a longitudinal axis and including: a. an outer tubular jacket; and b. an articulation member disposed within the jacket in the deflection region, the articulation member comprising a plurality of longitudinally-arranged tubular segments, a plurality of first connecting segments, a plurality of second connecting segments, and a beam; c. wherein: a. adjacent tubular segments are joined by respective ones of the first and second connecting segments; b. all of the first and second connecting segments are disposed in a first plane extending through the longitudinal axis; c. the beam extends in the first plane across the tubular segments and the first and second connecting segments; wherein a plurality of diametrically opposed slit pairs are disposed longitudinally along the articulation member, each slit pair separating adjacent tubular segments between respective ones of the first and second connecting segments and including a first slit and a second slit; and the slit pairs are centered on a second plane that is orthogonal to the first plane.

In Example 32, the medical device of Example 31, wherein the beam bisects a lumen in the articulation member into two sectors.

In Example 33, a medical device comprising: a. a handle; and b. a tubular shaft having a proximal portion extending from the handle, and a distal portion having a distal end and a deflection region, the shaft defining a longitudinal axis and including: a. an outer tubular jacket; and b. an articulation member disposed within the jacket in the deflection region, the articulation member having a tube that defines a plurality of openings and a beam extending across the tube, wherein: c. the openings are arranged in a first array and a second array; d. the openings in the first array are diametrically opposed to the openings in the second array; and e. the openings in the first array do not extend to the beam.

In Example 34, the medical device of Example 33, wherein the beam includes fillets where the beam connects to the tube.

In Example 35, the medical device of Example 34, wherein the openings do not extend into any of the fillets.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a heart of a patient using an electrophysiology system, consistent with various aspects of the present disclosure.

FIGS. 2A-2C are perspective views of a catheter, consistent with various aspects of the present disclosure. In FIG. 2A, the catheter is depicted in a straight configuration. In FIG. 2B, the catheter is depicted in a first deflected configuration. In FIG. 2C, the catheter is depicted in a second deflected configuration.

FIG. 3 is a side view of a deflection region of a shaft of the catheter, consistent with various aspects of the present disclosure.

FIGS. 4A-4F are views of an articulation member from the deflection region of the catheter, consistent with various aspects of the present disclosure. FIG. 4A is a top view of the articulation member. FIG. 4B is a cross-sectional view of the articulation member as indicated by line 4B-4B in FIG. 4A. FIG. 4C is a cross-sectional view of the articulation member as indicated by line 4C-4C in FIG. 4A. FIG. 4D is a side view of the articulation member. FIG. 4E is a perspective view of the articulation member. FIG. 4F is a cross-sectional view of the articulation member as indicated by line 4F-4F in FIG. 4E.

FIGS. 5A-5F are views of an alternative articulation member from the deflection region of the catheter, consistent with various aspects of the present disclosure. FIG. 5A is a side view of the alternative articulation member. FIG. 5B is a cross-sectional view of the alternative articulation member as indicated by line 5B-5B in FIG. 5A. FIG. 5C is a side view the articulation member, consistent with various aspects of the present disclosure. FIG. 5D is a side view the articulation member, consistent with various aspects of the present disclosure. FIG. 5E is a perspective view of the alternative articulation member. FIG. 5F is a cross-sectional view of the alternative articulation member as indicated by line 5F-5F in FIG. 5E.

FIG. 6 is a perspective view of an alternative articulation member, consistent with various aspects of the present disclosure.

FIG. 7 is a perspective view of an alternative articulation member, consistent with various aspects of the present disclosure.

FIG. 8 is a perspective view of an alternative articulation member, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

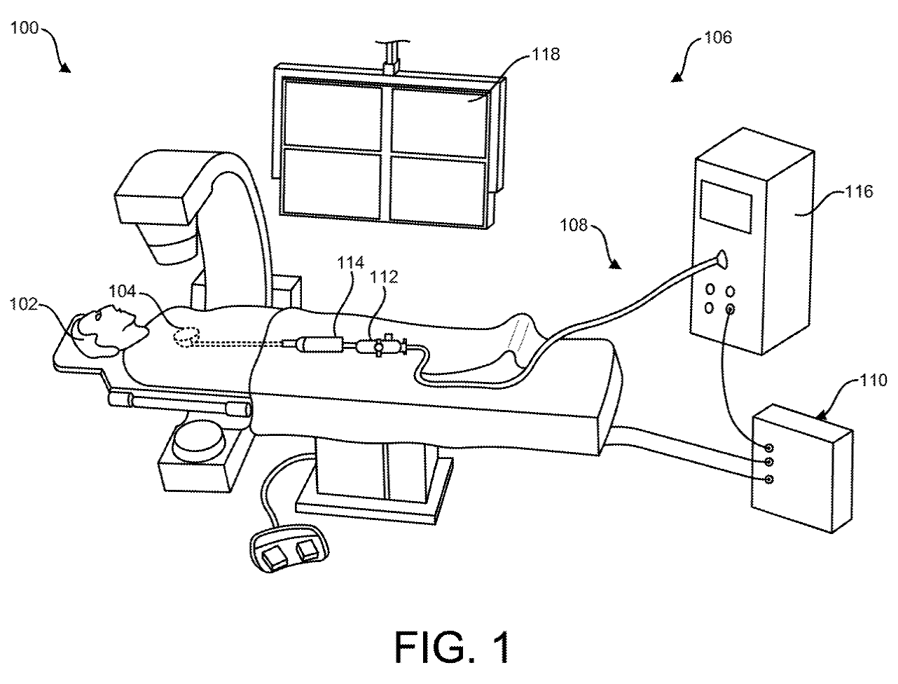

FIG. 1 illustrates an example clinical setting 100 for treating a patient 102, such as for treating a heart 104 of the patient 102, using an electrophysiology system 106. In the illustrated embodiment, the electrophysiology system 106 includes an electrophysiology catheter system 108 and an electro-anatomical mapping (EAM) system 110. The example electrophysiology catheter system 108 includes an electrophysiology catheter 112 (e.g., an electroporation catheter), an introducer sheath 114, and an electrophysiology console 116.

In some embodiments, the electrophysiology console 116 includes a controller, such one or more controllers, processors, or computers, that executes instructions or code, such as processor-executable instructions, out of a non-transitory computer readable medium, such as a memory device, or memory, to cause, such as control or perform, the aspects of the electrophysiology catheter system 108. Additionally, the electrophysiology catheter system 108 includes various connecting elements, such as cables, that operably connect the components of the electrophysiology catheter system 108 to one another and to the components of the EAM system 110.

In the illustrated embodiment, the introducer sheath 114 is operable to provide a delivery conduit through which the electrophysiology catheter 112 can be deployed to the specific target sites within the patient’s heart 104. Access to the patient’s heart 104 can be obtained through a vessel (not shown), such as a peripheral artery or vein. Once access to the vessel is obtained, the electrophysiology catheter 112 can be navigated to within the patient’s heart 104, such as within a heart chamber.

In the illustrated embodiment, the electrophysiology catheter system 108 can be configured to map and/or ablate portions of the patient’s heart 104. When ablating, the electrophysiology catheter system 108 is configured to deliver ablation electric field energy to targeted tissue in the patient’s heart 104 to create cell death in tissue, for example, rendering the tissue incapable of conducting electrical signals. When mapping, the electrophysiology catheter system 108 is configured to generate electric fields using the electrophysiology catheter 112 to create and present on a display 118, an electro-anatomical map of the patient’s heart 104. In some embodiments, the EAM system 110 includes the OPAL HDx™ mapping system marketed by Boston Scientific Corporation. In some embodiments, the mapping is performed using the INTELLAMAP ORION™ mapping catheter marketed by Boston Scientific Corporation. The mapping aids a physician in planning the ablation prior to delivering ablation electric field energy to the electrophysiology catheter 112.

The depiction of the electrophysiology system 106 shown in FIG. 1 is intended for illustration or a general overview of the various components of the system 106 and is not intended to imply that the disclosure is limited to any set of components or arrangement of the components. For example, additional hardware components, such as breakout boxes or workstations, can be included in the electrophysiology system 106.

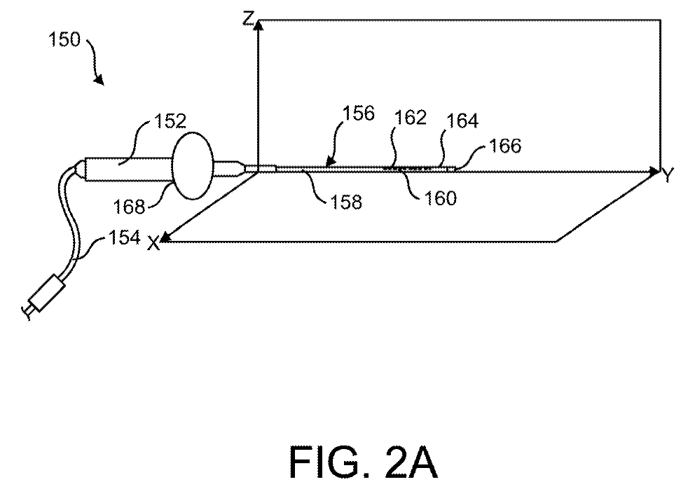

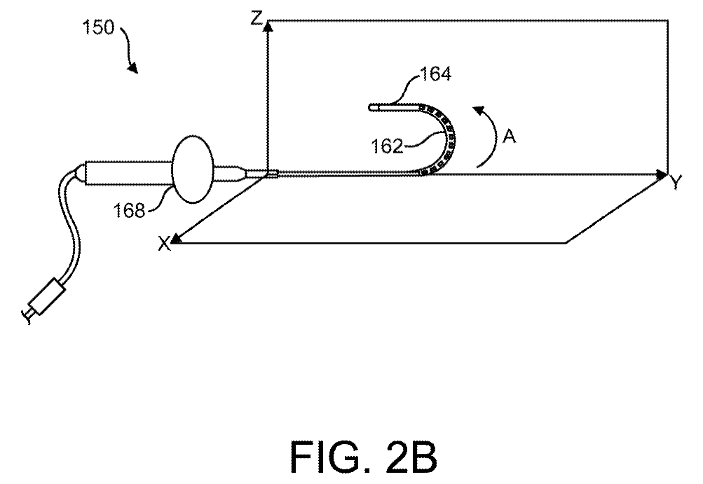

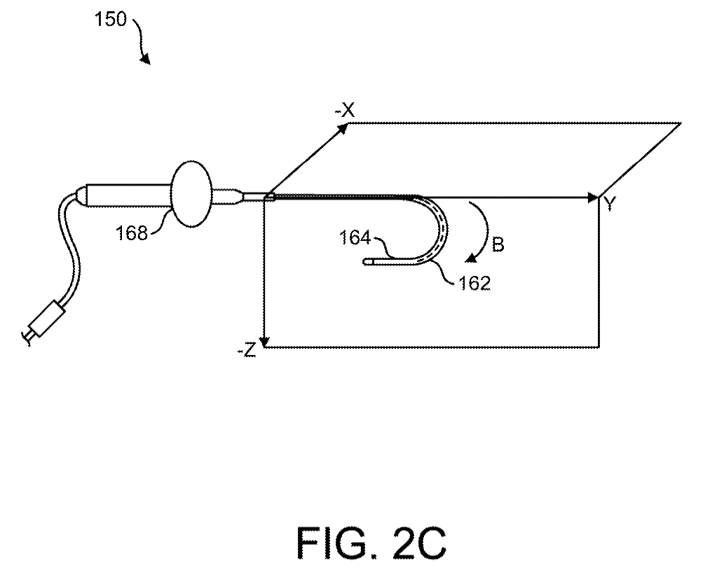

FIG. 2 shows a catheter 150 which can represent, for example, the electrophysiology catheter 112 (shown in FIG. 1). In the illustrated embodiment, the catheter 150 includes a handle 152, a connector 154 extending proximal from the handle 152, and a tubular shaft 156 extending distal from the handle 152. The shaft 156 includes a non-rigid, naturally straight proximal portion 158 and a deflectable distal portion 160, wherein the distal portion 160 is configured for placement and manipulation within a in a target area of a heart of the patient 102 (shown in FIG. 1). The distal portion 160 includes a deflection region 162 and an end region 164. In some embodiments, the deflection region 162 is about 10cm (4.0in.) long. The end region 164 is another straight section includes an electrode 166 that is located the distal end of the catheter 112. The connector 154 is selectively connectable to other equipment (e.g., the electrophysiology console 116, shown in FIG. 1) that can send or receive electrical signals to or from the electrode 116.

In the illustrated embodiment, the handle 152 includes an actuator 168 that is configured for steering the catheter 112. The physician can manipulate the actuator 168 to control the amount and direction of deflection of the deflection region 162. Such deflection can be analyzed using a Cartesian coordinate system. In an undeflected state (i.e., a straight configuration), the shaft 156 (i.e., the proximal portion 158 and the distal portion 160) extends along a Y-axis, as shown in FIG. 2A. Perpendicular to the Y-axis are the X-axis and the Z-axis, and in some embodiments, the deflection region 162 is deflectable in the YZ-plane but not in the XY-plane.

FIG. 2B shows the catheter 150 in a first deflected configuration. In the illustrated embodiment, the physician has manipulated the actuator 168 to deflect the deflection region 162 in a counterclockwise direction A so that the end region 164 is positively offset from the Y-axis and is generally oriented in the -Y direction. As will be explained below, the deflection of the catheter 150 occurs substantially solely in the YZ-plane and not substantially in the XY-plane. However, such uniplanar curvature can be affected by the vasculature of the patient 102 since the vasculature can impart forces to the sides of the catheter 150 that can move the bend out of plane.

FIG. 2C shows the catheter 150 in a second deflected configuration. In the illustrated embodiment, the physician has manipulated the actuator 168 to deflect the deflection region 162 in a clockwise direction B so that the end region 164 is negatively offset from the Y-axis and is generally oriented in the -Y direction. As will be explained below, the deflection of the catheter 150 occurs substantially solely in the YZ-plane and not substantially in the XY-plane. However, such uniplanar curvature can be affected by the vasculature of the patient 102 since the vasculature can impart forces to the sides of the catheter 150 that can move the bend out of plane.

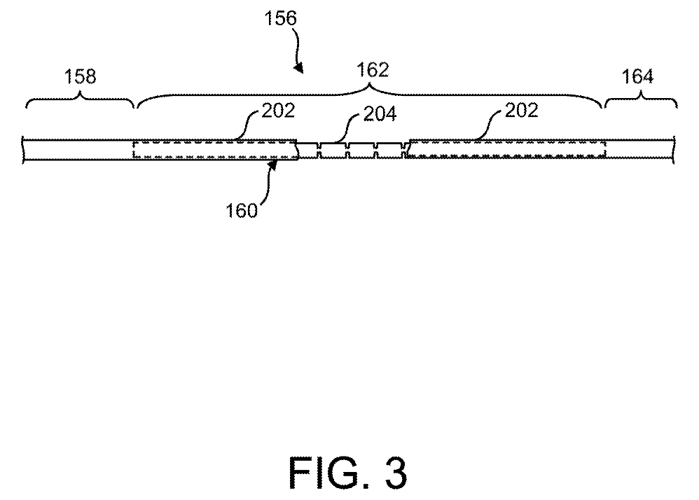

FIG. 3 is a side view of the shaft 156. In the illustrated embodiment, the shaft 156 includes an outer tubular jacket 202, which has been partially cut away to show an articulation member 204 inside of the jacket 202. In some embodiments, the jacket 402 is comprised of a braided polymer material reinforced with metal or polymeric wires that are woven, knitted, entwined, or otherwise interlaced together. In other embodiments, the jacket 202 is constructed differently, for example, using reinforcing coils to enhance the structural and torsional strength of the jacket 202.

In the illustrated embodiment, the articulation member 204 is positioned in the deflection region 162 which begins at the distal end of the proximal end portion 158 and extends to the proximal end of the end region 164. As will be explained in greater detail below, the articulation member 204 is configured to exhibit a relatively high degree of flexibility in the YZ-plane (shown in FIG. 2C), while at the same time being relatively inflexible in the XY-plane (shown in FIG. 2C). As such, the articulation member 204 facilitates predictable, highly planar deflection of the deflection region 162 by resisting torsional forces on the shaft 156 that would otherwise tend to cause the deflection region 162 to deflect or bend in the XY-plane (or some other plane oriented transversely to the YZ-plane).

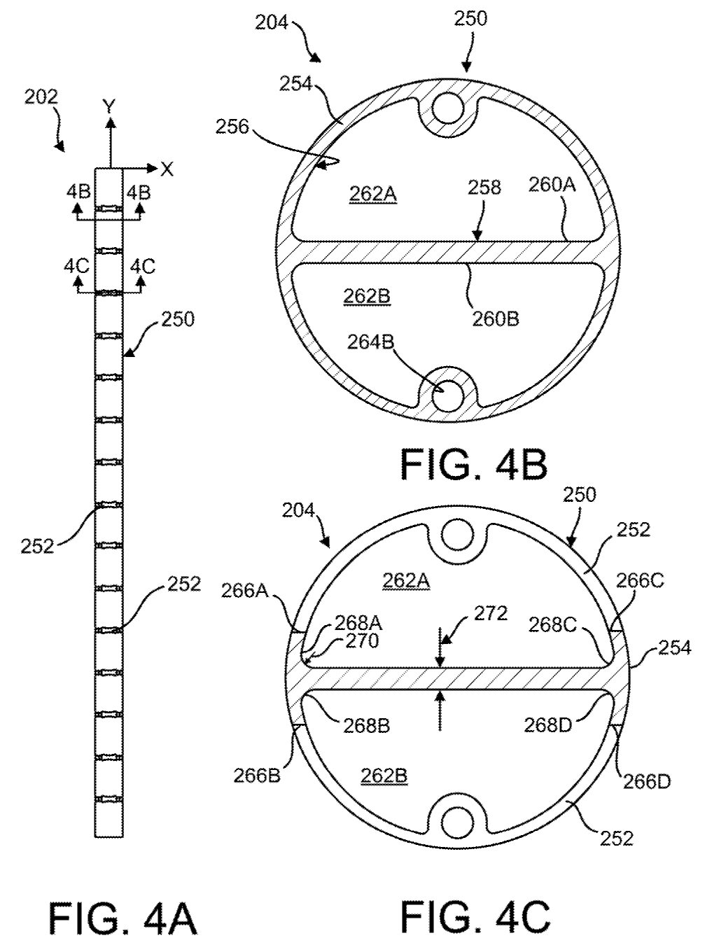

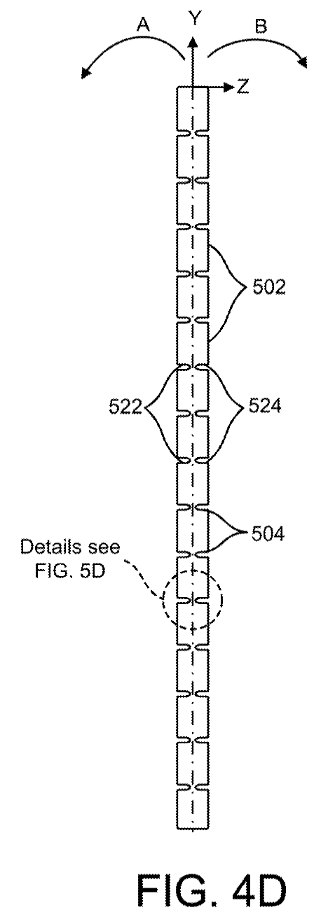

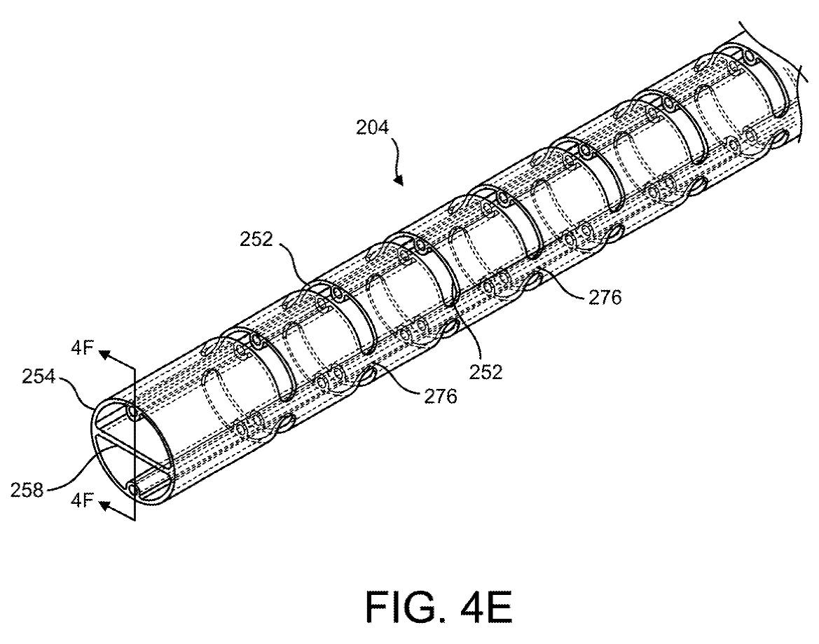

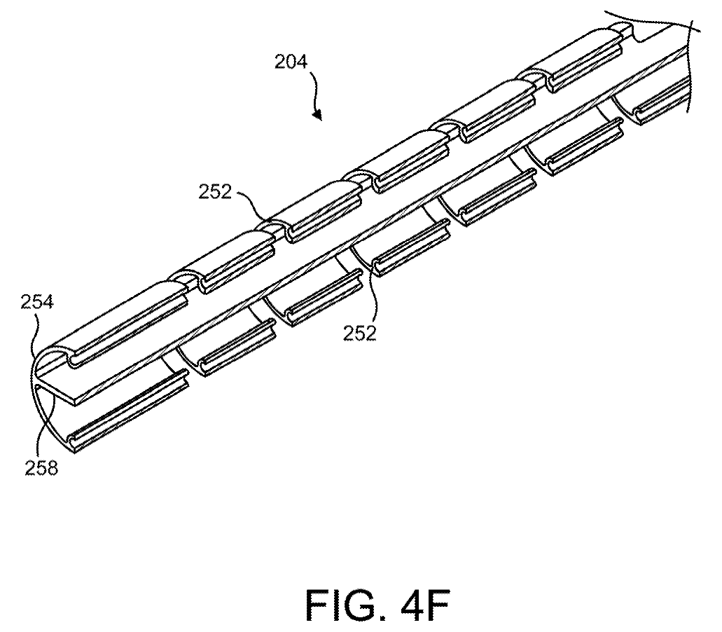

FIG. 4A shows a top view of the articulation member 204. In the illustrated embodiment, the articulation member 204 is a longitudinally extending tube 250 with an array of openings 252 defined by the tube 250. In some embodiments, the openings 252 are evenly longitudinally spaced apart from each other along the entire length of the tube 250. In other embodiments, the openings 252 extend along only part of the length of the tube 250 and/or are unevenly longitudinally spaced apart from each other. In some embodiments, all of the openings 252 are the same size and shape. In some embodiments, the openings 252 are U-shaped, V-shaped, bulb-shaped, inverted-T-shaped, and/or lollipop-shaped.

FIG. 4B shows a cross-sectional view of the articulation member 204. This cross-sectional view does not intersect any of the openings 252, so a wall 254 of the tube 250 forms a closed shape at this location. In the illustrated embodiment, the tube 250 comprises a compliant material, such as, for example, a polymer material (e.g., polyether ether ketone (PEEK), polyurethane (PU) (e.g., Pellethane®), polyimide (PI) (e.g., Aurum^{TM}), polypropylene (PP), or polycarbonate (PC)).

In the illustrated embodiment, the tube 250 includes a central lumen 256 arranged centrally with respect to the wall 254. The central lumen 256 is configured to allow the passage of other implements through the articulation member 204, such as, for example, wires for ablation electrodes, navigational components, temperature sensors (e.g., thermocouples), force sensors, radio-frequency circuitry and/or wires, and/or cooling lumens. In the illustrated embodiment, the tube 250 includes a beam 258 that extends like a chord from one circumferential (or perimetric) position on the wall 254 to another circumferential (or perimetric) position on the wall 254. In some embodiments, the two positions are diametrically opposed to one another, and in the illustrated embodiment, the beam 258 extends in the XY-plane. As such, the surfaces 260 (i.e., top surface 260A, bottom surface 260B) of the beam 258 are parallel to and offset from the XY-plane by equal and opposite distances. The beam 258 divides the central lumen 256 into two sectors 262 (i.e., sectors 262A, 262B). In some embodiments, the beam 258 bisects the central lumen 256 into two equally sized sectors 262. In other embodiments, the beam 258 is offset from the XY-plane in the Z-direction (shown in FIG. 2B) such that one of the sectors 262 is larger than the other one. In some embodiments, the beam 258 is a homogenous structure and/or solid body that does not include any lumens.

In the illustrated embodiment, the tube 250 also includes a pair of steering wire lumens 264 (i.e., lumens 264A, 264B) that are diametrically opposed to one another. The lumens 264 are positioned in the YZ-plane (shown in FIG. 2C), which is the plane in which the articulation member 204 primarily curves. Each of the lumens 264 are configured to accommodate a steering wire (not shown) that are connected to the actuator 168 (shown in FIG. 2C).

FIG. 4C shows a cross-sectional view of the articulation member 204. This cross-sectional view intersects two of the openings 252, so the wall 254 of the tube 250 forms an open shape at this location. The cross-section used in FIG. 4C shows the innermost extents of the openings 252, which are indicated by the edges 266 (i.e., edges 266A-266D) of the wall 254 that define the openings 252. In some embodiments, the articulation member 204 is manufactured starting with an extruded length of material that includes the lumens 256, 264 but not the openings 252. The openings 252 are formed by laser cutting across the material, which is why the edges 266 are oriented horizontally in FIG. 4C. In other words, the edges 266 are oriented parallel to and offset from the XY-plane (shown in FIG. 4A) instead of being, for example, radially oriented. Once all of the openings 252 are cut in the material, the tube 250 is complete. In other embodiments, the tube 250 is molded or additively manufactured with the openings 252 being included in the process.

In the illustrated embodiment, the beam 258 includes fillets 268 (i.e., fillets 268A-268D) where the beam 258 is connected to the wall 254. In some embodiments, the fillets 268 have a constant radius of curvature 270. In the illustrated embodiment, the ends of the fillets 268 are spaced apart from the innermost extent of the openings 252, which are indicated by the edges 262. As such, the openings 252 do not extend into the fillets 268 or to the beam 258. In the illustrated embodiment, a thickness of the beam 258 (i.e., the distance between the surfaces 260) is less than a vertical distance in the Z-direction (shown in FIG. 2B) between the ends of the fillets 268 and the edges 262. In some embodiments, the thickness 272 of the beam 258 is between one quarter of the radius of curvature 270 and four times the radius of curvature 270, or between one half of the radius of curvature 270 and twice the radius of curvature 270.

FIG. 4D shows a side view of the articulation member 204. One conception of the articulation member 204 is that it includes a plurality of longitudinally arranged tubular segments 274 that extend longitudinally (i.e., in the Y-direction) between the openings 252. The segments 274 are connected by respective ones of a plurality of living hinges 276 that exist laterally (i.e., in the Z-direction) between the openings 252 and longitudinally between the segments 274. In the illustrated embodiment, each living hinge 276 comprises a portion of the beam 258 (shown in FIG. 4C) and a two separate portions of the wall 254 (e.g., the hatched portions of the wall 254 shown in FIG. 4C). The largest portion of each living hinge 276 is the portion of the beam 258, so each living hinge 276 primarily extends in the XY-plane (shown in FIG. 4A). Thus, the living hinges 276 bend in the YZ-plane. Such bending causes elastic deformation of the living hinges 276 that results in elastic compression in one side of the living hinges 276 and elastic tension in the other side of the living hinges 276.

In the illustrated embodiment, the configurations of the openings 252, the tubular segments 274, and the living hinges 276 are uniform along the length of the articulation member 204. In other embodiments, some or all of the openings 252, some or all of the tubular segments 274, and/or some or all of the living hinges 276 have different configurations (e.g., with respect to spacing, lengths, and/or shapes), thus, enabling fine-tuning of the articulation of the deflection region 162 of the shaft 156 (shown in FIG. 3).

Such a design that incorporates the living hinges 276 enables the articulation member 204 to exhibit a relatively high degree of flexibility in the YZ-plane, while at the same time being relatively inflexible in the XY-plane. The plurality of openings 252 facilitate the articulation member 204 to have predictable, highly planar deflection of the deflection region 162 by resisting torsional forces on the shaft 156 that would otherwise tend to cause the deflection region 162 to deflect or bend in the XY-plane (or some other plane oriented transversely to the YZ-plane).

FIG. 4E is a perspective view of the articulation member. FIG. 4F is a cross-sectional view of the articulation member as indicated by line 4F-4F in FIG. 4E. FIGS. 4E and 4F will now be discussed in conjunction with one another.

In the illustrated embodiment, the articulation member 204 is manufactured from a continuous multi-lumen extrusion that is then cut (e.g., using a laser) to form the openings 252. In some embodiments, the openings 252 are substantially “U-shaped,” and in other embodiments, the openings 252 are substantially “V-shaped.”

In the illustrated embodiment, the beam 258 provides additional material (i.e., additional cross-sectional area) to the living hinges 276 compared to living hinges that only comprise two portions of the wall. Thus, the wall 254 is thinner compared to the wall of an articulation member without a beam, but the living hinges 276 are still able to maintain uniformity and planarity of the bending without plastic deformation. The wall 254 being thinner increased the cross-sectional area of the central lumen 256, which allows for an increased payload to travel through the articulation member 204 and be delivered to the target area of the patient 102 (shown in FIG. 1).

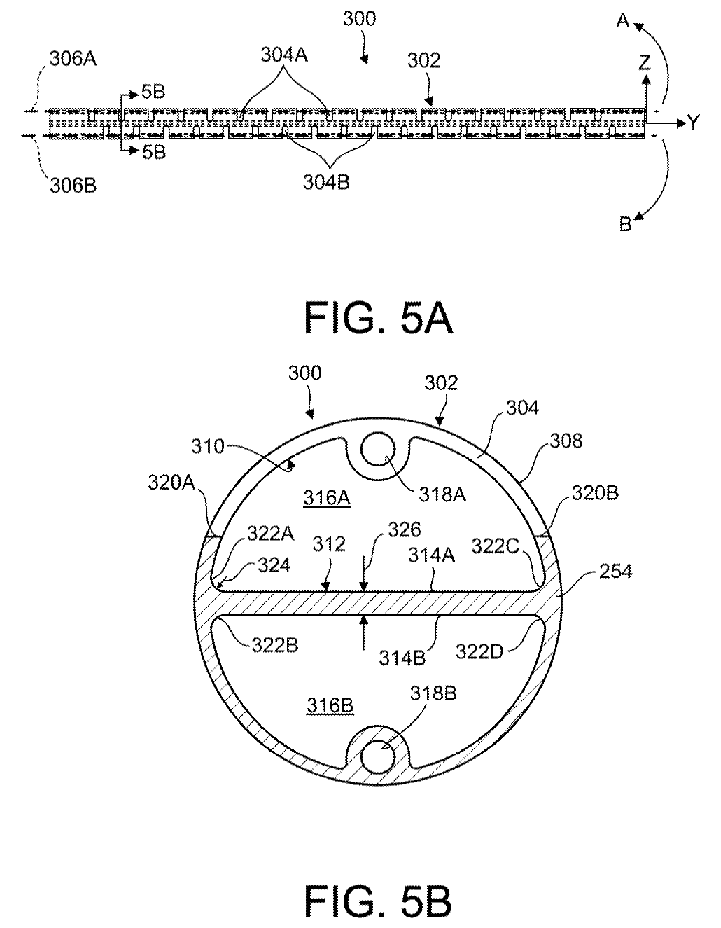

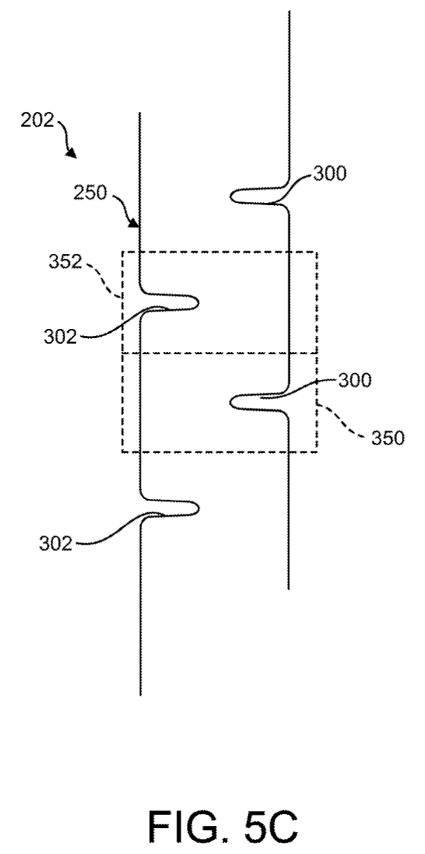

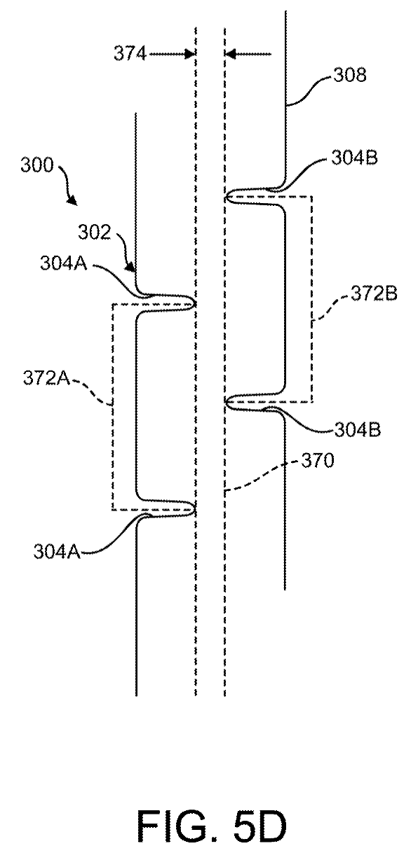

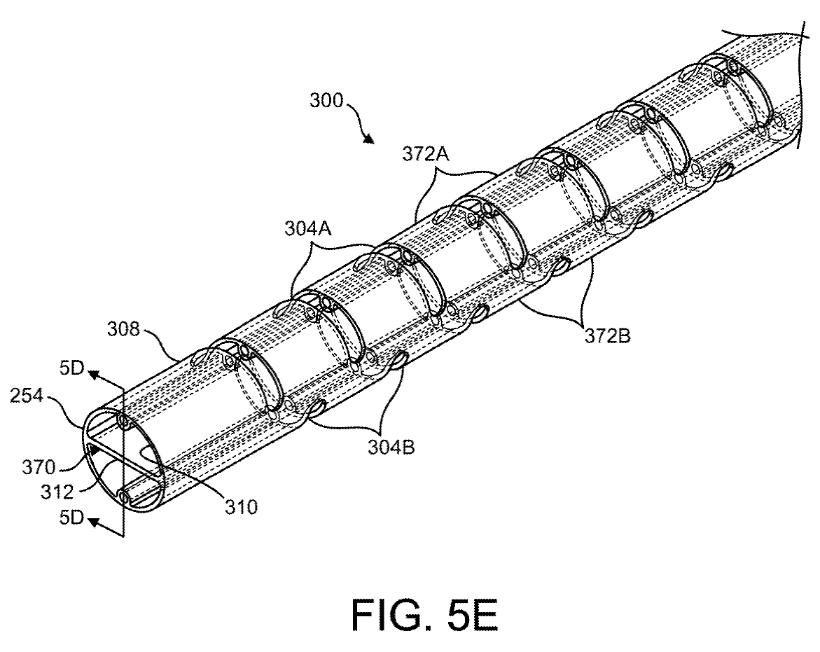

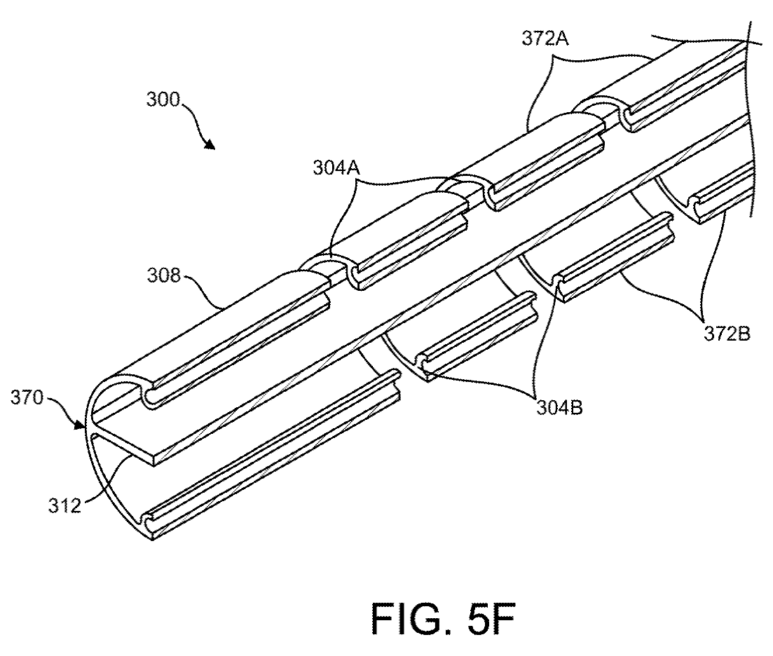

FIG. 5A is a side view of an alternative articulation member 300. In the illustrated embodiment, a tube 302 includes two sets of openings 304 (i.e., openings 304A, 304B) that are diametrically opposed to and longitudinally offset from one another. The arrangement of the openings 304 give the tube 302 a serpentine shape when viewed from the side. Specifically, there is an array of top openings 304A on the positive Z-axis side of the tube 302, and there is an array of bottom openings 304B on the negative Z-axis side of the tube 302. The array of top openings 304A is longitudinally offset from the array of bottom openings 304B. In some embodiments, there are the same number of top openings 304A and bottom openings 304B, although in other embodiments, there is one more of one of the openings 304A, 304B than the other of the openings 304A, 304B. In some embodiments, all of the openings 304 are the same size. In some embodiments, the top openings 304A are equally spaced apart from each other, and the bottom openings 304B are equally spaced apart from each other. In some embodiments, the top openings 304A are positioned halfway between the bottom openings 304B, and the bottom openings 304B are positioned halfway between the top openings 304A.

In the illustrated embodiment, the axes 306 (i.e., axes 306A and 306B) of the steering wire lumens (shown in FIG. 5B) extend longitudinally through the tube 302 in the YZ-plane, and the axes 306 are parallel to and equally offset from the Y-axis in opposite directions. The steering wire lumens are configured to receive steering wires that facilitate the deflection of at least the deflection region 162 (shown in FIG. 3) in a conventional manner. For example, when the steering wire that extends along the axis 304B is pulled by the actuator 168 (shown in FIG. 2C), that steering wire pulls on one side of the tube 302. The tension causes the top openings 304A to expand and the bottom openings 304B to contract, and the articulation member 300 bends in the clockwise direction B. For another example, when the steering wire that extends along the axis 304A is pulled by the actuator 168, that steering wire pulls on one side of the tube 300. The tension causes the bottom openings 304B to expand and the top openings 304A to contract, and the articulation member 300 bends in the counterclockwise direction A.

FIG. 5B shows a cross-sectional view of the articulation member 300. This cross-sectional view intersects one of the openings 304A, so a wall 308 of the tube 302 forms an open shape at this location. In the illustrated embodiment, the tube 302 comprises a compliant material, such as, for example, a polymer material (e.g., polyether ether ketone (PEEK), polyurethane (PU) (e.g., Pellethane®), polyimide (PI) (e.g., Aurum^{TM}), polypropylene (PP), or polycarbonate (PC)).

In the illustrated embodiment, the tube 302 includes a central lumen 310 arranged centrally with respect to the wall 308. The central lumen 310 is configured to allow the passage of other implements through the articulation member 300, such as, for example, wires for ablation electrodes, navigational components, temperature sensors (e.g., thermocouples), force sensors, radio-frequency circuitry and/or wires, and/or cooling lumens. In the illustrated embodiment, the tube 302 includes a beam 312 that extends like a chord from one circumferential position on the wall 308 to another circumferential position on the wall 308. In some embodiments, the two positions are diametrically opposed to one another, and in the illustrated embodiment, the beam 312 extends in the XY-plane. As such, the surfaces 314 (i.e., top surface 314A, bottom surface 314B) of the beam 312 are parallel to and offset from the XY-plane by equal and opposite distances. The beam 312 divides the central lumen 310 into two sectors 316 (i.e., sectors 316A, 316B). In some embodiments, the beam 312 bisects the central lumen 310 into two equally sized sectors 316. In other embodiments, the beam 316 is offset from the XY-plane in the Z-direction (shown in FIG. 5A) such that one of the sectors 316 is larger than the other one. In some embodiments, the beam 312 is a homogenous structure and/or solid body that does not include any lumens.

In the illustrated embodiment, the tube 302 also includes a pair of steering wire lumens 318 (i.e., lumens 318A, 318B) that are diametrically opposed to one another. The lumens 318 are positioned in the YZ-plane (shown in FIG. 5A), which is the plane in which the articulation member 300 primarily curves. Each of the lumens 318 are configured to accommodate a steering wire (not shown) that are connected to the actuator 168 (shown in FIG. 2C).

The cross-section used in FIG. 5B shows the innermost extents of the openings 304A, which are indicated by the edges 320 (i.e., edges 320A, 320B) of the wall 308 that define the openings 304A. While not shown, the openings 304B are similar to or the same as the openings 304A, albeit with an opposite orientation with respect to the XY-plane (shown in FIG. 2B). In some embodiments, the articulation member 300 is manufactured starting with an extruded length of material that includes the lumens 310, 318 but not the openings 304. The openings 304 are formed by laser cutting across the material, which is why the edges 320 are oriented horizontally in FIG. 5B. In other words, the edges 320 are oriented parallel to and offset from the XY-plane instead of being, for example, radially oriented. Once all of the openings 304 are cut in the material, the tube 302 is complete. In other embodiments, the tube 302 is molded or additively manufactured with the openings 304 being included in the process.

In the illustrated embodiment, the beam 312 includes fillets 322 (i.e., fillets 322A-322D) where the beam 312 is connected to the wall 308. In some embodiments, the fillets 322 have a constant radius of curvature 324. In the illustrated embodiment, the ends of the fillets 322 are spaced apart from the innermost extent of the openings 304, which are indicated by the edges 320. As such, the openings 304 do not cut into the fillets 322. In the illustrated embodiment, a thickness of the beam 312 (i.e., the distance between the surfaces 314) is less than a vertical distance in the Z-direction (shown in FIG. 5) between the ends of the fillets 322 and the edges 320. In some embodiments, the thickness 326 of the beam 312 is between one quarter of the radius of curvature 324 and four times the radius of curvature 324, or between one half of the radius of curvature 324 and twice the radius of curvature 324.

FIG. 5C shows a close-up side view the articulation member 300. As discussed earlier with respect to FIG. 5B, the articulation member 300 can be thought of as the tube 302 with openings 304 cut into it. However, a different way of understanding the articulation member 300 is as a longitudinally extending array of living hinges 350, 352 with alternating orientations along the length of the tube 302. The living hinges 350 are oriented in one direction (e.g., to the right in FIG. 5C), and the living hinges 352 are oriented in the opposite direction (e.g., to the left in FIG. 5C). In the illustrated embodiment, each living hinge 350 includes a portion of the tube 302 with one of the openings 300, and each living hinge 352 includes a portion of the tube 302 with one of the openings 302. Thus, each living hinge 350, 352 has an opposite orientation from the adjacent living hinge(s) 350, 352.

In the illustrated embodiment, when the articulation member 300 is actuated, each of the living hinges 350 will become more opened or more closed, and each of the living hinges 352 will do the opposite from the living hinges 350. The opening and closing of the living hinges 350, 352, respectively, allow the central lumen 310 (shown in FIG. 5B) to remain open as the articulation member 300 deflects.

The opposing orientations (i.e., 180° apart) of the living hinges 350, 352 means that the deflection of the articulation member 300 occurs in only the deflection plane (e.g., the YZ-plane, shown in FIG. 5A). At the same time, the articulation member 300 is relatively inflexible in the orthogonal plane (e.g., the XY-plane, shown in FIG. 2B). As such, the living hinges 350, 352 facilitate the articulation member 300 to have predictable, highly planar deflection of the deflection region 162 (shown in FIG. 3) by resisting torsional forces on the articulation member 300 that would otherwise tend to cause the deflection region 162 to deflect or bend in, for example, the XY-plane (or some other plane oriented transversely to, for example, the YZ-plane). In some embodiments, the living hinges 350, 352 are configured to maintain the deflection of the articulation member 300 within ±10° of the deflection plane under normal conditions in the clinical setting (shown in FIG. 1).

FIG. 5D shows a close-up side view the articulation member 300. As discussed earlier with respect to FIG. 5B, the articulation member 300 can be thought of as the tube 302 with openings 304 cut into it. However, yet another different way of understanding the articulation member 300 is as a longitudinally extending rail 370 that includes the beam 312 (shown in FIG. 5B) and two diametrically opposed portions of the wall 308 (so only one portion of the wall 308 that is included in the rail 370 is visible in FIG. 5D). Thus, the rail 370 has an “H-shaped” or “I-shaped” cross-section. The rail 370 is joined on alternating sides by cylindrical shell sectors 372 (i.e., sectors 372A, 372B). In the illustrated embodiment, the rail 370 is a longitudinally straight, continuous section that extends between the innermost extents of the openings 304, which is possible because the openings 304 do not extend past the center of the tube 302. In other words, the openings 304 do not circumferentially overlap each other. In some embodiments, the width 374 of the rail 370 at the wall 308 is the same on both sides and is between about 0.35mm (0.014in.) and about 2.0mm (0.080in.), between about 0.51mm (0.020in.) and about 2.0mm (0.080in.), or between about 0.35mm (0.014in.) and about 1.5mm (0.059in.). In some such embodiments, the diameter of the shaft 156 (shown in FIG. 3) is about 2.834mm (8.5 Fr).

In the illustrated embodiment, the cylindrical shell sectors 372 have slightly less than a half-pipe shape. The rail 370 and the cylindrical shell sectors 372A define the openings 304A, and the rail 370 and the cylindrical shell sectors 372B define the openings 304B. Thus, the sectors 372A are separated from each other by the openings 304A, and the sectors 372B are separated from each other by the openings 304B. The cylindrical shell sectors 372A are diametrically opposed to and longitudinally offset from the cylindrical shell sectors 372B. Thus, each sector 372A longitudinally overlaps two adjacent sectors 372B, and each sector 372B longitudinally overlaps two adjacent sectors 372A (although at the last sectors 372 at the ends of the articulation member 300 may longitudinally overlap only one of the opposite sectors 372, respectively).

When the articulation member 300 deflects, the rail 370 bends. In the illustrated embodiment, if the deflection is to the right, then the bending of the rail 370 causes the adjacent sectors 372B to move closer together (or perhaps to contact each other) and the adjacent sectors 372A to move farther apart. In such a scenario, the sides of the rail 370 that are closest to the sectors 372B are under a compressive load and the sides of the rail 370 that are closest to the sectors 372A are under a tensile load. In the illustrated embodiment, if the deflection is to the left, then the bending of the rail 370 causes the adjacent sectors 372A to move closer together (or perhaps to contact each other) and the adjacent sectors 372B to move farther apart. In such a scenario, the sides of the rail 370 that are closest to the sectors 372A are under a compressive load and the sides of the rail 370 that are closest to the sectors 372B are under a tensile load. Such compressive and tensile loads cause deformation of the rail 370 as the articulation member 300 deflects.

In the various embodiments, the longitudinal staggering of the openings 304, combined with configuring the openings 304 such that they do not overlap circumferentially, provides an articulating member that exhibits increased resistance to undesired plastic deformation at the hinge portions as compared to existing articulation member configurations in which the openings are longitudinally aligned and/or circumferentially overlap one another.

FIG. 5E is a perspective view of the alternative articulation member 300. FIG. 5F is a cross-sectional view of the alternative articulation member 300 as indicated by line 5F-5F in FIG. 5E. FIGS. 5E and 5F will now be discussed in conjunction with one another.

In the illustrated embodiment, the configurations of the openings 304, the cylindrical shell sectors 372, and the rail 370 are uniform along the length of the articulation member 300. In other embodiments, some or all of the openings 304, some or all of the cylindrical shell sectors 372, and/or the rail 370 have different configurations (e.g., with respect to spacing, lengths, and/or shapes), thus, enabling fine-tuning of the articulation of the deflection region 162 of the shaft 156 (shown in FIG. 3).

In the illustrated embodiment, the articulation member 300 is manufactured from a continuous multi-lumen extrusion that is then cut (e.g., using a laser) to form the openings 304. In some embodiments, the openings 304 are substantially “U-shaped,” and in other embodiments, the openings 304 are substantially “V-shaped.”

In the illustrated embodiment, the beam 312 provides additional material (i.e., additional cross-sectional area) to the articulation member 300 compared to an articulation member that only comprises a wall. Thus, the wall 308 is thinner compared to the wall of an articulation member without a beam, but the articulation member 300 is still able to maintain uniformity and planarity of the bending without plastic deformation. The wall 308 being thinner increased the cross-sectional area of the central lumen 310, which allows for an increased payload to travel through the articulation member 300 and be delivered to the target area of the patient 102 (shown in FIG. 1).

Such a design that incorporates the features shown in FIGS. 5A-5F enables the articulation member 300 to exhibit a relatively high degree of flexibility in the YZ-plane, while at the same time being relatively inflexible in the XY-plane. The plurality of openings 304 facilitate the articulation member 300 to have predictable, highly planar deflection of the deflection region 162 by resisting torsional forces on the shaft 156 that would otherwise tend to cause the deflection region 162 to deflect or bend in the XY-plane (or some other plane oriented transversely to the YZ-plane).

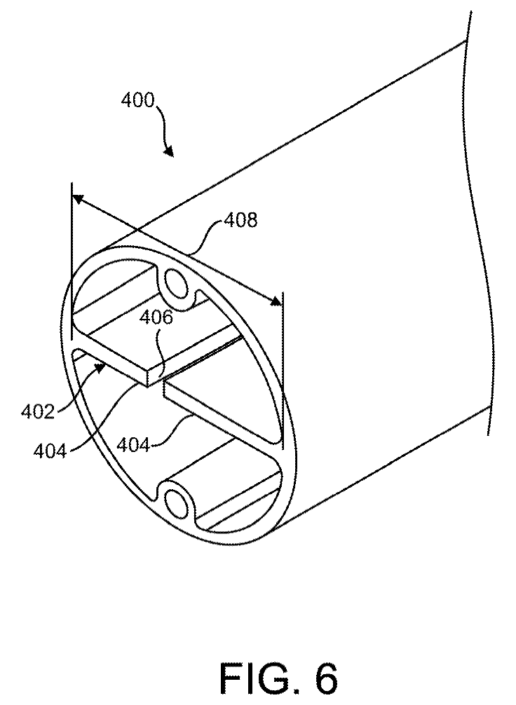

FIG. 6 is a perspective view of an alternative articulation member 400. In the illustrated embodiment, the articulation member 400 includes a beam 402 that is similar to the beams 258, 312 (shown in FIGS. 4F, 5F, respectively) except that the beam 402 comprises two “T-shaped” spars 404 each extending radially inward and toward each other, with a gap 406 therebetween. In some embodiments, the spars 404 have similar or the same size and shape. In some embodiments, the width of the gap 406 is less than about 50%, less than about 25%, or less than about 10% of a width 408 of the beam 402.

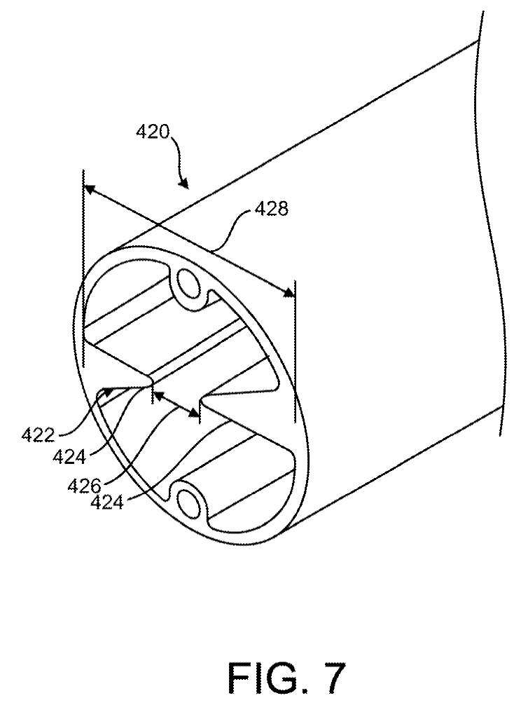

FIG. 7 is a perspective view of an alternative articulation member 420. the illustrated embodiment, the articulation member 420 includes a beam 422 that comprises two spars 424 with a gap 426 therebetween. Such an arrangement is similar that of the beam 402 with spars 404 (shown in FIG. 6), however, the spars 424 are tapered down towards the gap 426. In some embodiments, the spars 424 have similar or the same size and shape. In some embodiments, the width of the gap 426 is less than about 50%, less than about 25%, or less than about 10% of a width 428 of the beam 422.

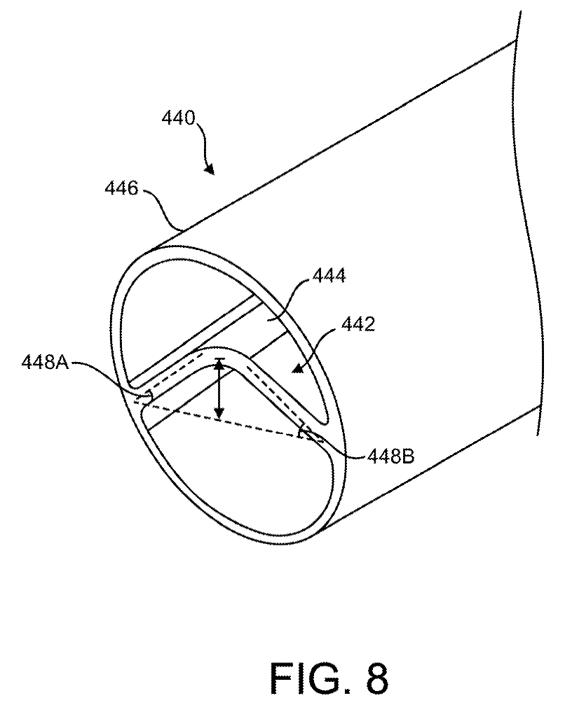

FIG. 8 is a perspective view of an alternative articulation member 440. the illustrated embodiment, the articulation member 440 includes a beam 442 that is similar to beams 258, 312 (shown in FIGS. 4F, 5F, respectively) except that the beam 442 includes a bend 444. Thus, the beam 442 connects to a wall 446 of the articulation member 440 at angles 448 (i.e., angles 448A and 448B). In some embodiments, the bend 444 is centrally located, so the angles 448 are the same. In some embodiments, the angles 448 are between about 5° and about 35°, between about 10° and about 30°, or between about 15° and about 25°. Such a configuration of the beam 442 means that there is more material toward one circumferential position than the opposite circumferential position. For example, in FIG. 8, more of the beam 442 exists on the upper side of the articulation member 440 than on the lower side of the articulation member 440. In some embodiments, depending on the material characteristics, the articulation member 440 will bend more easily toward the side with less of the beam 442. In such embodiments, the radius of curvature of the articulation member 440 towards that side will be smaller than the radius of curvature of the articulation member 440 towards the side with more of the beam 442.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical device comprising:
a handle; and
a tubular shaft having a proximal portion extending from the handle, and a distal portion having a distal end and a deflection region, the shaft defining a longitudinal axis and including:
an outer tubular jacket; and
an articulation member disposed within the jacket in the deflection region, the articulation member having a wall and a beam extending across the wall from a first perimetric position to a second perimetric position, wherein:
the tube defines a plurality of openings;
the openings are arranged in a first array and a second array;
the openings in the first array are diametrically opposed to the openings in the second array; and
the openings in the first array do not circumferentially overlap the beam.

2. The medical device of claim 1, wherein the beam bisects a lumen in the articulation member into two sectors.

3. The medical device of claim 2, wherein the two sectors are equally-sized.

4. The medical device of claim 1, wherein the beam includes fillets where the beam connects to the wall.

5. The medical device of claim 4, wherein the openings do not extend into any of the fillets.

6. The medical device of claim 4, wherein a radius of curvature of the fillets is between one-quarter and four times a thickness of the beam.

7. The medical device of claim 1, wherein the first perimetric position is diametrically opposed to the second perimetric position.

8. The medical device of claim 1, wherein a first surface of the beam is parallel to a second surface of the beam.

9. The medical device of claim 1, wherein the beam comprises two spars with a gap therebetween.

10. The medical device of claim 1, wherein the beam includes a bend such that the beam is connected to the wall at an angle.

11. The medical device of claim 1, wherein the first array is longitudinally offset from the second array.

12. The medical device of claim 11, wherein the first array is offset such that an opening in the first array is longitudinally positioned halfway between two openings in the second array.

13. The medical device of claim 1, wherein the articulation member includes a first steering wire lumen on a first side of the beam and a second steering wire lumen on a second side of the beam.

14. The medical device of claim 13, wherein:
the first steering wire lumen and the second steering wire lumen are positioned in a first plane;
the beam extends in a second plane; and
the first plane is orthogonal to the second plane.

15. The medical device of claim 1, wherein an innermost extent of the plurality of openings is parallel to the beam.

16. A medical device comprising:
a handle; and
a tubular shaft having a proximal portion extending from the handle, and a distal portion having a distal end and a deflection region, the shaft defining a longitudinal axis and including:
an outer tubular jacket; and
an articulation member disposed within the jacket in the deflection region, the articulation member comprising a plurality of longitudinally-arranged tubular segments, a plurality of first connecting segments, a plurality of second connecting segments, and a beam;
wherein:
adjacent tubular segments are joined by respective ones of the first and second connecting segments;
all of the first and second connecting segments are disposed in a first plane extending through the longitudinal axis;
the beam extends in the first plane across the tubular segments and the first and second connecting segments; wherein a plurality of diametrically opposed slit pairs are disposed longitudinally along the articulation member, each slit pair separating adjacent tubular segments between respective ones of the first and second connecting segments and including a first slit and a second slit; and the slit pairs are centered on a second plane that is orthogonal to the first plane.

17. The medical device of claim 16, wherein the beam bisects a lumen in the articulation member into two sectors.

18. A medical device comprising:
a handle; and
a tubular shaft having a proximal portion extending from the handle, and a distal portion having a distal end and a deflection region, the shaft defining a longitudinal axis and including:
an outer tubular jacket; and
an articulation member disposed within the jacket in the deflection region, the articulation member having a tube that defines a plurality of openings and a beam extending across the tube, wherein:
the openings are arranged in a first array and a second array;
the openings in the first array are diametrically opposed to the openings in the second array; and
the openings in the first array do not extend to the beam.

19. The medical device of claim 18, wherein the beam includes fillets where the beam connects to the tube.

20. The medical device of claim 19, wherein the openings do not extend into any of the fillets.
